(19) United States
(12) Patent Application Publication
(10) Pub. No.: US 2025/0188399 A1
(43) Pub. Date: Jun. 12, 2025

(54) BIOREACTOR

(71) Applicant: Ivy Farm Technologies Limited, Oxford, Oxfordshire (GB)

(72) Inventors: Kiruthika Lawton, Oxford (GB); Robin Nyland, Oxford (GB); Simon William Partridge, Oxford (GB); Akinlolu Odeleye, Oxford (GB)

(21) Appl. No.: 18/837,162

(22) PCT Filed: Feb. 8, 2023
(86) PCT No.: PCT/GB2023/050283

§ 371 Date: Aug. 8, 2024

(30) Foreign Application Priority Data
Feb. 8, 2022 (GB) 2201608.3

(51) Int. Cl. C12M 1/12; C12M 3/00; C12M 3/04

(52) U.S. Cl. CPC C12M 25/14; C12M 21/08; C12M 27/10

(57) ABSTRACT

There is provided a method of culturing cells, wherein the method comprises: placing a substrate in a bioreactor, wherein the substrate is in the form of a rolled-up film; culturing cells on the substrate in the biore-actor; and optionally, removing the substrate from the bioreactor; wherein the substrate is processed using roll-to-roll processing and, as part of the roll-to-roll processing, at least one of the following steps occur: a plurality of channels are introduced into the substrate; a surface roughness of the substrate is increased; a coating is applied to the substrate; the substrate is subjected to a surface treatment step; cultured cells are removed from the substrate. Also provided is a bioreactor system for culturing cells comprising a substrate within the bioreactor, wherein the substrate is in the form of a rolled-up film and the rolled-up film has a plurality of channels.

### FIELD

The present invention relates to a method of culturing cells. The present invention further relates to a bioreactor system.

### BACKGROUND OF THE INVENTION

There are concerns about the sustainability of traditional farming practices relating to the rearing of livestock. These traditional farming practices are energy intensive, land intensive and utilise a large amount of antibiotics. A possible approach for addressing these concerns is the culture of meat in a bioreactor. This requires far less energy and land and is a desirable approach for feeding the world's growing population.

However, the success of meat culture depends on the commercial viability of the culture process. The present invention provides means for increasing meat culture viability and efficiency.

### SUMMARY OF THE INVENTION

The present invention provides a method of culturing cells, wherein the method comprises the following steps: (i) placing a substrate in a bioreactor, wherein the substrate is in the form of a rolled-up film; (ii) culturing cells on the substrate in the bioreactor; and (iii) optionally, removing the substrate from the bioreactor; wherein the substrate is processed using roll-to-roll processing and, as part of the roll-to-roll processing, at least one of the following steps occur: (a) a plurality of channels are introduced into the substrate; (b) a surface roughness of the substrate is increased; (c) a coating is applied to the substrate; (d) the substrate is subjected to a surface treatment step; (e) cultured cells are removed from the substrate.

The present invention further provides a method of preparing a bioreactor, wherein the method comprises the following steps: (i) placing a substrate in a bioreactor, wherein the substrate is in the form of a rolled-up film; wherein the substrate is processed using roll-to-roll processing and, as part of the roll-to-roll processing, at least one of the following steps occur: (a) a plurality of channels are introduced into the substrate; (b) a surface roughness of the substrate is increased; (c) a coating is applied to the substrate; (d) the substrate is subjected to a surface treatment step.

Incorporating roll-to-roll processing of the substrate into the process of culturing cells enables an efficient process. In particular, the rolled-up form of the substrate means that the substrate can be directly taken from a roll-to-roll process, or subsequently utilised in a roll-to-roll process, without the need for any further processing steps, as well as providing the advantage of an increased amount of surface area on which to culture cells during the cell culture process.

The present invention can be generally used for the culture of cells, in particular adherent cells (cells that grow on a substrate, also known as anchorage-dependent cells). The invention is particularly advantageous for culturing muscle and/or fat cells for the production of meat for consumption, e.g. human consumption. Thus, the cells introduced (seeded) into the bioreactor may be capable of differentiating into myocytes (including myotubes) and/or adipocytes. In particular, the cells introduced into the bioreactor may be stem cells, progenitor cells, or precursor cells. Exemplary cells include embryonic stem cells (ESCs), induced pluripotent stem cells (iPSCs), mesenchymal stem cells (MSCs), muscle stem cells (muscle satellite cells), myoblasts, pre-adipocytes, or combinations thereof. Myoblasts are particularly preferred. The cells introduced into the bioreactor may be primary cells or cell lines. Exemplary cell lines include Chinese Hamster Ovary (CHO) cells and C2C12 cells (a myoblast subclone).

Although, the cells may be seeded in the bioreactor, it can be advantageous to additionally or alternatively seed the cells on to the substrate before the substrate is introduced into the bioreactor. For example, the cells may be seeded on to the substrate as part of the roll-to-roll processing. This can occur in addition or as an alternative to any of the other roll-to-roll processing steps that are described herein.

During cell culture in the bioreactor, the cells or a portion of the cells may differentiate into myocytes (including myotubes) and/or adipocytes (including white fat cells and brown fat cells). Myocytes (including myotubes) are particularly preferred. Thus, the invention may be used for differentiating cells.

### DETAILED DESCRIPTION OF THE INVENTION

Cells for use in the invention may be of any animal origin. However, typically, the cells are not human cells. In particular, preferably, the cells are not produced through the destruction of human embryos. The cells are typically cells of non-human animals, such as non-human mammals, birds, fish, crustaceans, molluscs, reptiles, amphibians, or insects. Exemplary non-human mammals include those in the genera Bovinae, Camelidae, Canidae, Caprae, Cervidae, Felidae, Equidae, Lagomorphs, Macropodidae, Oves, Rodents, or Suidae. The cells may be cells of livestock or poultry. The cells may be porcine, bovine, ovine, caprine, avine, or piscine.

The bioreactor that can be used with the present invention is any suitable bioreactor for culturing adherent cells. In general terms, a bioreactor is a vessel that can support the maintenance and growth of cells. This is also referred to as the culture of cells.

The bioreactor houses the substrate along with cell culture medium, and optionally cells. The cell culture medium may contain nutrients and growth factors within a fluid that supports the maintenance, proliferation, and growth of the cells. The cell culture medium may contain factors promoting cell differentiation. An exemplary culture medium is Dulbecco's Modified Eagle Medium (DMEM). Cell culture medium for use in the invention may comprise serum or may not comprise serum. The cells that are adhered on the substrate are submerged in the cell culture medium. The cell culture medium may be moved around the bioreactor to facilitate the growth of the cells.

A particularly preferred approach is to use perfusion of the cell culture medium. Perfusion refers to the introduction of medium into the bioreactor while removing medium from the bioreactor. It is particularly preferred that the new medium is introduced at the bottom of the bioreactor and the spent medium is removed from the top of the bioreactor (top and bottom are in the sense that top is above bottom along the direction of gravity).

The new medium can be introduced by being pumped into the bioreactor. The spent medium may be removed by being pumped out of the bioreactor. The introduced medium may be fresh medium, in the sense it has not been previously introduced into the bioreactor and the removed medium can be spent medium, in the sense that it has been exposed to growing cells. However, the perfusion approach encompasses recirculating, at least part of, the medium through the bioreactor.

The bioreactor can regulate the temperature of the medium within which the cells are growing. This allows optimization of the cell growth.

The bioreactor may be any suitable size for culturing the cells. The invention is particularly useful for large scale reactors. The approach efficiently processes a high area substrate improving the overall efficiency of the cell culture process. Accordingly, the bioreactor may have a capacity of at least 5 litres or at least 10 litres, preferably at least 20 litres, even more preferably at least 50 litres.

The substrate can be any material that can be used in a bioreactor that is suitable for culturing adherent cells on its surface. The substrate has to be formed from a material that is capable of adopting the configuration of a rolled-up film, and be suitable for being processed in roll-to-roll processing.

The rolled-up configuration is a configuration of the material where it is wound around an axis a plurality of times. This can be achieved by rolling it around itself or rolling it around a spindle. This results in a layered structure, where the layers are crossed as you move from the central winding axis to the outer surface of the substrate. In the present invention there is space between adjacent layers of the rolled-up form. This allows the culture medium to circulate around the substrate surfaces within the rolled-up configuration when the substrate is placed in the bioreactor. This allows growth of cells throughout the substrate. The rolled-up form advantageously provides a large amount of surface area for a given volume.

The substrate may be roll-to-roll processed. Roll-to-roll processing of the substrate, as used herein, refers to a process where the substrate is unwound and rewound into the rolled-up form and a processing step is conducted as part of this process. For example, the substrate is unwound from one spindle, and rolled-up on another spindle, while the material moving between the spindles is subjected to a coating process. This is a particularly advantageous approach for use with the present invention given the advantages associated with using the substrate in its rolled-up form for culturing cells.

Suitable materials for the rolled-up film include polymeric materials, such as poly (α-hydroxy acids), polyurethanes, and polysaccharides. A preferred material comprises, consists essentially of, or consists of poly (α-hydroxy acid). A particularly preferred material comprises, consists essentially of, or consists of polyethylene terephthalate (PET). PET has been found to be particularly useful for growing cells and implementing other features of the present invention.

The term “film” as used herein refers to a material that has a relatively thin thickness dimension compared to its length and width dimensions. In particular, the thickness of the film is sufficiently thin so as to allow the material to adopt the rolled up configuration. The film may be less than 1 cm, or less than 5 mm, or less than 2 mm, or less than 1 mm thick. The film may have a length of over 1 metre, or over 5 metres, or over 10 metres, or over 20 metres. The film may have a width of more than 30 cm, or more than 50 cm. In the rolled-up configuration, the film is rolled up along its length. In the rolled-up configuration, the film may be rolled about itself at least 5 times, preferably at least 10 times, preferably at least 20 times.

The conditions for culturing cells are any suitable conditions that will result in the growth of cells. Conditions that can be adjusted include the composition of the culture medium, the temperature within the bioreactor, the pH of the culture medium, along with any circulating, agitation, or perfusion settings.

The substrate can be placed in the bioreactor before the introduction of the cells to the bioreactor. Alternatively, cells may be introduced (seeded) onto the substrate before the substrate is placed into the bioreactor. In other words, the substrate may already have cells attached to its surface when it is placed into the bioreactor.

The substrate may be removed from the bioreactor after cell culture. However, it is possible to retain the substrate within the bioreactor between culture batches when the cells are removed from the substrate without requiring its removal from the bioreactor. For example, the cells may be removed from the substrate using chemical methods or an enzyme and so reduce the need to remove the substrate from the bioreactor.

The substrate may be utilised for multiple cell culturing and cell removal cycles. This makes efficient use of the substrate by minimising the number of processing steps.

The substrate within the bioreactor may be just one rolled-up film. Alternatively, a plurality of rolled-up films may be introduced into the bioreactor. In particular, the plurality of rolled-up films may be stacked on top of each other. They may be stacked such that the longitudinal axis (the axis around which the rolled-up film is wound) of each rolled-up film is aligned. This may be an advantageous configuration for a particularly long (tall) bioreactor.

As noted above, the invention advantageously incorporates roll-to-roll processing. This increases the efficiency of the culture process. The roll-to-roll processing may be used for any number of processing steps, in particular any number or combination of the roll-to-roll processing steps described herein. The use of roll-to-roll processing allows processing to occur continuously along the length of the substrate material. This is particularly advantageous for scaling-up the cell culture process.

The roll-to-roll processing may be used for forming a plurality of channels in the substrate. These channels are introduced into the substrate before the substrate is placed in the bioreactor. The term “channel” refers to a continuous region that is bound by surface features. For example, a relatively depressed region running along the surface that is bound by relatively protruding regions would be considered a channel in accordance with the present invention. It is particularly preferred that the plurality of channels are in the form of ridges and valleys. In this way the valley regions are channels that are bound by the ridge regions. The plurality of channels can be of the form of a repeating pattern. For example, the plurality of channels can be formed from alternating ridge and valley regions. It is noted that the definition of a ridge and the definition of a valley may depend on the perspective from which they are viewed. For example, a film that has been formed to have a relatively raised region on one surface may have a correspondingly relatively depressed region when the opposing surface is viewed.

When the substrate is in the rolled-up configuration the channels assist in maintaining a distance between adjacent layers and thus allow the culture medium to flow over more of the substrate surface.

The channels may run continuously across the rolled-up film. Preferably, the channels run continuously along the width of the film. In this way, the channels provide a continuous path for the culture medium to flow over the surface of the substrate. This is particularly advantageous when the bioreactor is a perfusion bioreactor as the flow path of the cell culture medium can be arranged so that the cell culture medium flows along the channels. All of the channels may run along the substrate in parallel.

The channels may be introduced into the film by running the substrate through meshing gears as part of the roll-to-roll processing. This process is particularly advantageous when the film comprises PET, since PET can be readily deformed by this process.

The meshing gears deform the film plasticly so that it conforms to the shape defined by the meshing gears. Accordingly, the channels are shaped such that they approximate to the shape of the meshed gears. The gears are separated by a distance that allows the film to run between the gears while imparting a force on both sides of the film so as to form the channels in the film.

The gears may be made of any suitable material. For example, the gears may be made from metal and, in particular, stainless steel. The gears are shaped in accordance with the desired channel shape for the film. The gears extend preferably along a length that is greater than the width of the film. This allows one set of gears to impart the required channels along the full width of the film as the film runs along its length direction through the meshing gears.

The plurality of channels may be of any suitable size to allow the cell culture medium and the cells to move through the channels. For example, the channels may have a depth of 0.1 mm to 10 mm. The depth may be considered to be the distance between the relatively highest point on a surface in the raised region and the lowest relative height on that surface in the depressed region. The channels may have a repeat distance of between 1 and 100 mm. The channels may have a repeat distance of between 1 and 50 mm. The repeat distance is the distance between corresponding points in a repeating pattern of channels, for example the distance between the highest point on adjacent raised sections. A shallower channel will assist in increasing the amount of surface area that will fit in the bioreactor by allowing the layers to pack closer together, while a deeper channel may assist in maintaining a high flow rate of cell culture medium by reducing the resistance to fluid flow. The channels may also be sized to act as alignment guides for the cells, for example myoblasts. In this way, the channels may be used to facilitate myogenesis (myotube formation). This advantageously allows the substrate to support the proliferation of the cells and the differentiation of the cells into myotubes.

A surface roughness of the substrate may be increased as part of the roll-to-roll processing. The surface roughness of the substrate is increased before the substrate is placed in the bioreactor. The surface roughness may be increased by applying an abrasive material to the surface of the substrate. For example, the surface roughness may be increased by applying sandpaper to the substrate. The grit size of the sandpaper may be between 600 and 320, i.e. the average particle diameter on the sandpaper is between 16 micrometres to 36 micrometres. A particularly preferable grit size is 400 (23 micrometre average particle diameter). The application of the abrasive material can occur on the film as it passes between the two rolls on the roll-to-roll processing setup. In this manner the surface roughness may be increased in an efficient manner. Overall, increasing the surface roughness can increase the surface area of the substrate and assist in maximising the adherence of cells onto the substrate.

The abrasive material preferably introduces grooves into the substrate. The introduced grooves include grooves that are at least 5 microns in depth. The introduced grooves may be at least 5 microns in width. The grooves may be introduced so that they are substantially aligned with any channels that may be present.

A coating may be applied to the substrate as part of the roll-to-roll processing. The coating may be applied to the substrate before placing the substrate in the bioreactor. The coating of the substrate as part of the roll-to-roll process efficiently introduces the coating as part of utilising the rolled-up film.

The coating may comprise a peptide for enhancing attachment (adherence) of the cells to the substrate. The peptide may be, or may be derived from, an extracellular matrix (ECM) protein, such as laminin. Alternatively, the peptide may be a synthetic peptide.

The coating may comprise a thermoresponsive polymer. A thermoresponsive polymer is one that changes properties on temperature change. For example, the thermoresponsive polymer may change volume significantly on temperature change. As another example, a thermoresponsive polymer may exhibit hydrophilic/hydrophobic changes in response to temperature. Such changes in the polymer may be utilised to remove (detach) the cells from the substrate, for example at the end of the culture process. A thermoresponsive polymer that can be used with the present invention is poly (N-isopropylacrylamide).

The substrate may be subjected to a surface treatment step as part of the roll-to-roll processing. For example, the substrate may be subjected to plasma treatment and/or a UV treatment step. This can assist with subsequent cell adhesion to the substrate.

The cultured cells may be removed from the substrate. This may be done whilst the substrate is still inside the bioreactor or after the substrate has been removed from the bioreactor. The cultured cells may be removed using an enzymatic process, for example by trypsinization. The cultured cells may be removed using a chemical process, for example by treatment with Ca- and Mg-free PBS and EDTA. The cultured cells may be removed using a mechanical process. The cultured cells may be removed from the substrate after the substrate is removed from the bioreactor. In particular, the substrate may be removed from the bioreactor and may undergo roll-to-roll processing in order to remove the cultured cells.

The cultured cells may be removed from the substrate by contacting the substrate with a tool during the roll-to-roll processing. This can mechanically remove the cells from the substrate in an efficient manner. The tool may have an edge that contacts the substrate in order to scrape along the substrate and so remove the cells.

Once removed from the substrate, the cells may be used directly as the meat product for consumption. Alternatively, the cells may be biomass which is further processed into a final meat product for consumption. Thus, the methods of the invention may further comprise a step of processing the cells into a meat product for consumption, e.g. for human consumption.

A number of different process steps that can be conducted during roll-to-roll processing are described herein. Any number of these processing steps can be combined together in the present invention. For example, roll-to-roll processing before the substrate is placed in the bioreactor may be used to coat the substrate and introduce the channels as well as increase the surface roughness. Also, roll-to-roll processing may be used to remove the cultured cells from the substrate after the substrate is removed from the bioreactor following culture of the cells.

The present invention also provides a bioreactor system for culturing cells. The bioreactor system comprises a bioreactor, wherein the bioreactor has a longitudinal axis; and a substrate within the bioreactor, wherein the substrate is in the form of a rolled-up film and the rolled-up film has a plurality of channels, wherein each of the plurality of channels has a longitudinal axis and the longitudinal axes of the plurality of channels are substantially parallel to the bioreactor longitudinal axis.

The longitudinal axis of the bioreactor runs along its length. For example, when the bioreactor is cylindrical, the longitudinal axis runs along the length of the cylinder and perpendicular to its circular cross-section.

The longitudinal axis of each of the plurality of channels runs along the length of the respective channels.

As noted above the longitudinal axis of the bioreactor may be parallel to each of the longitudinal axes of the plurality of channels. This orients the channels to be aligned along the length of the bioreactor and so they are aligned along the common flow path for cell culture medium in the bioreactor. In this regard, when the bioreactor is a perfusion bioreactor, the inlet and outlet are preferably positioned at opposite ends of the longitudinal axis of the bioreactor.

The bioreactor system may be utilised in a method of the present invention. In particular, the bioreactor system may be utilised in a method comprising the following steps: introducing cells into the bioreactor (also referred to as seeding the bioreactor); maintaining the bioreactor such that the bioreactor longitudinal axis is at a first angle relative to the vertical; rotating the bioreactor around its longitudinal axis and further maintaining the bioreactor longitudinal axis at a second angle relative to the vertical; maintaining the bioreactor such that the bioreactor longitudinal axis is at a third angle relative to the vertical while culturing the cells on the substrate in the bioreactor, wherein the third angle is less than the first angle and less than the second angle.

By maintaining the bioreactor at a first angle relative to the vertical and then rotating the bioreactor around its longitudinal axis and maintaining it at a second angle relative to the vertical, the distribution of the cells within the bioreactor can be made more uniform. Subsequently, the bioreactor is moved to a third angle which results in the longitudinal axis moving closer to vertical. This assists in moving cell culture medium (containing the cells) through the bioreactor along the bioreactor longitudinal axis by utilising the effect of gravity to aid in the distribution of the cell culture medium and cells within the bioreactor, which in turn aids the distribution of the cells on the substrate. This is particularly advantageous when the inlet for perfusion of the bioreactor is at one end of the longitudinal axis of the bioreactor and the outlet for the perfusion of the bioreactor is at the opposite end of the longitudinal axis of the bioreactor. In this way, the channels are also aligned more with the flow direction of the cell culture medium.

The vertical is defined as the direction of gravitational force that may be illustrated by the direction along which a plumb line will hang.

The first angle may be substantially the same as the second angle. In this manner the angle does not need to be changed during the initial distribution of the introduced cells. Instead, the bioreactor is simply rotated in order to improve the distribution of the initial cells that are introduced into the bioreactor.

The third angle may be substantially zero. In other words, the longitudinal axis may be substantially vertical while culturing the cells on the substrate in the bioreactor. The first and second angle may be greater than 40°, or greater than 60°, or preferably greater than 75°. This allows a more even distribution of cells following their initial introduction into the bioreactor.

To aid distribution of the cells on the substrate, rotation through the first, second, and third angles should generally be carried out before the cells have adhered to the substrate, at least before the majority of cells have adhered to the substrate. Therefore, rotation through the first, second, and third angles may be carried out within 24, 18, 12, or 6 hours of introducing the cells into the bioreactor. Preferably, the rotations are performed at intervals of from 20 minutes to 3 hours, even more preferably at intervals of 1 to 2 hours.

The cultured cells may be used as a replacement for livestock-derived meat in a meat product for consumption using suitable food production techniques as are well known to the skilled person. For example, the cultured cells may be simply added into a mixture intended to form meatballs or sausages as a replacement or addition to livestock-derived meat, and the mixture then processed into the meat product using conventional techniques.

As used herein, the term “polymer” refers to oligomers and both homopolymers and copolymers, and the prefix “poly” refers to two or more.

Singular encompasses plural and vice versa. For example, although reference is made herein to “a” particulate substrate, “a” flow distributor, and the like, one or more of each of these and any other components can be used.

The terms “comprising” and “comprises” as used herein are synonymous with “including”, “includes” or “containing”, “contains”, and are inclusive or open-ended and do not exclude additional, non-recited members, elements or method steps.

Additionally, although the present invention has been described in terms of “comprising”, the invention as detailed herein may also be described as “consisting essentially of′ or “consisting of”.

Although the present invention has been described in terms of “obtainable by”, the associated features of the present invention detailed herein may also be independently described as “obtained by”.

As used herein, the term “and/or,” when used in a list of two or more items, means that any one of the listed items can be employed by itself or any combination of two or more of the listed items can be employed. For example, if a list is described as comprising group A, B, and/or C, the list can comprise A alone; B alone; C alone; A and B in combination; A and C in combination, B and C in combination; or A, B, and C in combination.

Where ranges are provided in relation to a genus, each range may also apply additionally and independently to any one or more of the listed species of that genus.

Attention is directed to all papers and documents which are filed concurrently with or previous to this specification in connection with this application and which are open to public inspection with this specification, and the contents of all such papers and documents are incorporated herein by reference.

All of the features disclosed in this specification (including any accompanying claims, abstract and drawings), and/or all of the steps of any method or process so disclosed, may be combined in any combination, except combinations where at least some of such features and/or steps are mutually exclusive.

Each feature disclosed in this specification (including any accompanying claims, abstract and drawings) may be replaced by alternative features serving the same, equivalent or similar purpose, unless expressly stated otherwise. Thus, unless expressly stated otherwise, each feature disclosed is one example only of a generic series of equivalent or similar features.

The invention is not restricted to the details of the foregoing embodiment(s). The invention extends to any novel one, or any novel combination, of the features disclosed in this specification (including any accompanying claims, abstract and drawings), or to any novel one, or any novel combination, of the steps of any method or process so disclosed.

The invention will now be described in relation to the following Figures and examples.

### FIGURES

FIG. 1 depicts a schematic illustration of the intermeshing gears introducing channels into the PET sheet;

FIG. 2 depicts the channelled PET film in its rolled-up configuration within an open bioreactor; and

FIG. 3 depicts confluent growth of cells on samples taken from six different layers of a channelled PET film roll.

### EXAMPLES

The biocompatibility of PET as culture substrate was tested. Commercially available PET sheets were cut into disks and air plasma treated for 3 minutes. The disks were then placed at the bottom of a 24 well plate and seeded with primary porcine myoblasts at 4000 cells/cm2. 98% cell adherence was observed within 24 h and the population doubling time was 58.3 hours. The cells were left in culture for 7 days and then detached from the PET sheets through enzymatic methods (trypsinization) and counted.

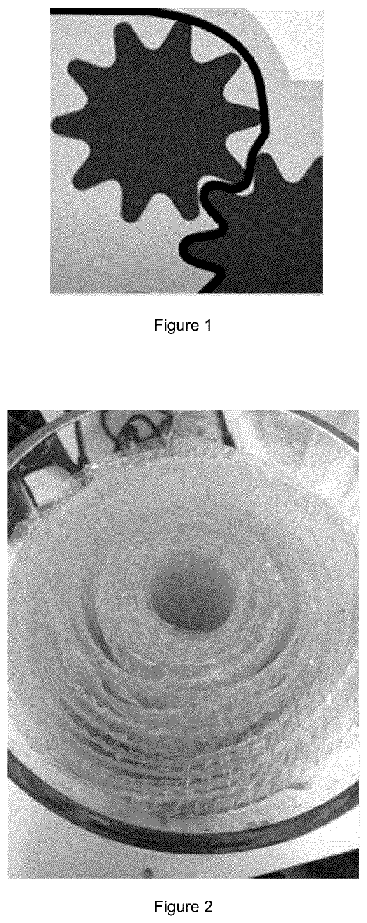

A PET sheet was obtained commercially having a thickness of approximately 0.190-0.200 mm. Grooves were produced on the PET sheets by passing them through a belt sander as part of a roll-to-roll process. The belt sander was equipped with 400 grit sandpaper. This produced grooves along the length direction of the sheet due to this being the direction of travel. Following this, perfusion channels were added by passing the PET sheet through two intermeshing gears (FIG. 1). The teeth of the gears had a peak-to-peak distance of 4.1 mm and a depth between teeth of 0.5 mm. This process embosses the sheet, which acts as perfusion channels. The sheets are then rolled up into a rolled-up form and placed inside the bioreactor, as illustrated in FIG. 2.

Initially, the PET sheets were embossed only on the outer parts of its width, leaving the central section without embossed channels. It was observed that the flow of the media was not uniform through the different layers which was due to the spacing between the different layers of the roll. When fluid is perfused through irregular spacing, fluid flow takes the path of least resistance. The roll was then embossed along its entire width of the sheet to create uniform spacing between the different layers of the roll along the full length. This was observed to enhance flow distribution.

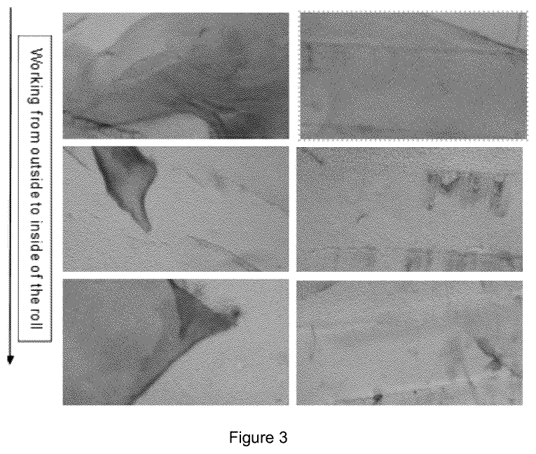

Cell growth on the various layers of the rolled-up configuration of the channelled PET was then assessed. This could also confirm that the channels supported flow distribution through the different layers and therefore cells were able to proliferate. The channelled PET sheets were plasma treated to increase the surface charge of the sheets. The results of this experiment are detailed in Table 1. After 7 days of culture in the bioreactor, samples were taken from six different layers of the PET roll and imaged using a light microscope. It was observed that the cells had reached confluency across all samples and were detaching as cell sheets (FIG. 3).

TABLE 1

| Parameter | Value | |
|---|---|---|
| Surface area | 450 | cm 2 |
| Density (cells/cm²) | 100000 | |
| Cells seeded | 45 | million |
| ECM coating (μg/cm²) | 0.5 or 0.05 | |
| Culture duration (days) | 7 | |
| Cell yield after 7 days of | 340 | million |

culture

A larger rolled-up form of the PET sheet was also tested. This allowed a relatively large surface area (28000 cm2) to be incorporated into a 3 litre bioreactor. The initial cell load introduced into the reactor was 2.9×108 cells. These were cultured for 7 days in the bioreactor, which led to 2.74×109 cells and a harvested cell mass of 3.02 g.

1. A method of culturing cells, wherein the method comprises the following steps:
(i) placing a substrate in a bioreactor, wherein the substrate is in the form of a rolled-up film;
(ii) culturing cells on the substrate in the bioreactor; and
(iii) optionally, removing the substrate from the bioreactor;
wherein the substrate is processed using roll-to-roll processing and, as part of the roll-to-roll processing, at least one of the following steps occur:
(a) a plurality of channels are introduced into the substrate;
(b) a surface roughness of the substrate is increased;
(c) a coating is applied to the substrate;
(d) the substrate is subjected to a surface treatment step;
(e) cultured cells are removed from the substrate.

2. The method of claim 1, wherein the plurality of channels are introduced into the substrate before step (i).

3. The method of claim 1, wherein the channels are introduced into the substrate by the film being run through meshing gears as part of the roll-to-roll processing.

4. The method of claim 1, wherein the plurality of channels are in the form of parallel ridges and valleys.

5. The method of claim 1, wherein the surface roughness of the substrate is increased before step (i).

6. The method of claim 1, wherein the surface roughness of the substrate is increased by applying an abrasive material to the surface of the substrate.

7. The method of claim 1, wherein the coating is applied to the substrate before step (i) and/or the substrate is subjected to a surface treatment step before step (i).

8. The method of claim 1 wherein the coating comprises a peptide for enhancing attachment of the cells to the substrate or a thermoresponsive polymer.

9. The method of claim 1, wherein the cultured cells are removed from the substrate after step (iii).

10. The method of claim 1, wherein the cultured cells are removed from the substrate by contacting the substrate with a tool during the roll-to-roll processing.

11. A bioreactor system for culturing cells comprising
a bioreactor, wherein the bioreactor has a longitudinal axis; and
a substrate within the bioreactor, wherein the substrate is in the form of a rolled-up film and the rolled-up film has a plurality of channels, wherein each of the plurality of channels has a longitudinal axis and the longitudinal axes of the plurality of channels are substantially parallel to the bioreactor longitudinal axis.

12. A method of culturing cells utilising the bioreactor system of claim 11, wherein the method comprises the following steps in order:
introducing cells into the bioreactor;
maintaining the bioreactor such that the bioreactor longitudinal axis is at a first angle relative to the vertical;
rotating the bioreactor around its longitudinal axis and further maintaining the bioreactor such that the bioreactor longitudinal axis is at a second angle relative to the vertical;
maintaining the bioreactor such that the bioreactor longitudinal axis is at a third angle relative to the vertical while culturing cells on the substrate in the bioreactor, wherein the third angle is less than the first angle and less than the second angle.

13. The method of claim 12, wherein the first angle is substantially the same as the second angle.

14. The method of claim 1 wherein the film is a poly (α-hydroxy acid) film, optionally a polyethylene terephthalate film.

15. The method of claim 1 wherein the cells introduced into the bioreactor comprise cells capable of differentiating into myocytes and/or adipocytes.

16. The method of claim 1, wherein the method further comprises a step of processing the cells into a meat product for consumption.

17. (canceled)
